(19) United States
(12) Patent Application Publication
(10) Pub. No.: US 2013/0178477 A1
(43) Pub. Date: Jul. 11, 2013

(54) METHODS OF TREATING DIABETIC NEUROPATHY USING BENZENESULFONAMIDES

(71) Applicant: AbbVie Inc., North Chicago, IL (US)

(72) Inventors: Qingwei Zhang, Grayslake, IL (US); Andrew O. Stewart, Libertyville, IL (US); Zhiren Xia, Gurnee, IL (US); Wolfram V. Nothaft, Riverwoods, IL (US)

(73) Assignee: AbbVie Inc., North Chicago, IL (US)

(21) Appl. No.: 13/734,385

(22) Filed: Jan. 4, 2013

Related U.S. Application Data
(60) Provisional application No. 61/583,391, filed on Jan. 5, 2012.

(51) Int. Cl. A61K 31/4985

(52) U.S. Cl. CPC A61K 31/4985
USPC 514/249

(57) ABSTRACT

This invention is directed to methods of treating diabetic neuropathy, particularly, diabetic peripheral neuropathy, in a patient in need of treatment thereof.

### FIELD

This invention is directed to methods of using benzenesulfonamides, particularly methods of treating diabetic neuropathy in patients of need thereof, using said benzenesulfonamides.

### BACKGROUND

Pain of various different types afflicts virtually all humans and animals at one time or another. A substantial number of medical disorders and conditions produce some sort of pain as a prominent concern requiring treatment. Pain is the one of the most common significant medical issues reported in the clinic and it affects the broadest group of patients. Distinct types and manifestations of pain are reported. Different pain types can be related to the origin of the pain, the underlying pathology, or different pharmacological agents that demonstrate efficacy (or lack thereof) in treating the pain.

The pain can be caused by different reasons. For example, the prominent causes of inflammatory pain are burns and chemical irritation. Post-surgical pain can arise after incisions of the skin and internal organs, among other conditions. Nerve damage can lead to neuropathic pain and neuropathy arising from sciatic nerve irritation, chronic diabetes, and chemotherapy.

Various types of pain are treated with distinct, different therapeutic agents. For example, acute inflammatory pain is typically treated with non-steroidal anti-inflammatory (NSAID) agents and cyclooxygenase inhibitors, for example aspirin or celecoxib. Post-surgical pain is typically treated with opiate receptor agonists, for example codeine. Neuropathic pain can be treated with anti-depressant agents and neurotransmitter reuptake inhibitors, for example duloxetine. Diabetic neuropathy pain is commonly treated with acetominophen.

A distinct type of pain where patients are not completely treatable by any of the currently available drugs or by agents is neuropathic pain. The drug duloxetine can usefully provide partial relief of this type of pain. Neuropathic pain can develop in response to previous injury or ongoing tissue injury, nerve injury, or diabetes. Neuropathic pain is distinct from other types of pain, for example inflammatory pain, in that it persists long after signs of the original injury or damage have disappeared. Neuropathic pain also is associated with allodynia, hyperalgesia, or causalgia (Dworkin Clinical Journal of Pain (2002) vol. 18(6) pp. 343-9). The topic and physiology of neuropathic pain has been reviewed in the scientific literature (Smith, et al. Drug Development Research (2001) vol. 54(3), pp. 140-153; Collins and Chessell Expert Opinion on Emerging Drugs (2005) vol. 10(1), pp. 95-108; Vinik and Mehrabyan Medical Clinics of North America (2004), vol. 88(4), pp. 947-999; Dray, Urban, and Dickenson Trends in Pharmacological Sciences (1994) vol. 15(6) pp. 190-7; Dworkin Clinical Journal of Pain (2002) vol. 18(6) pp. 343-9)

T-Type calcium channels represent a novel target for pain modulation. Over the last several years, a body of evidence has emerged indicating that activation of this channel contributes to ongoing chronic pain (See, Todorovic, S M, Jevtovic-Todorovic V. Regulation of T-type calcium channels in the peripheral pain pathway. Channels. 2007; 1(4):238-45 and Zamponi G W, Lewis R J, Todorovic S M, Arneric S P, Snutch T P. Role of voltage-gated calcium channels in ascending pain pathways. Brain Res Rev. 2009; 60(1):84-9). Cav3.2 is the predominant T-Type calcium channel in sensory nerves that modulate nociception (See, Chen C C, Lamping K G, Nuno D W, Barresi R, Prouty S J, Lovoie J L, et al. Abnormal coronary function in mice deficient in alpha1H T-type Ca2+ channels. Science. 2003; 302(5649):1416-8). Cav3.2 is expressed in DRG neurons, peripheral receptive fields, spinal cord dorsal horn, and brain (See, Talley E M, Cribbs L L, Lee J H, Daud A, Perez-Reyes E, Bayliss D A. Differential distribution of three members of a gene family encoding low voltage-activated (T-type) calcium channels. J. Neurosci. 1999; 19(6):1895-911). Data from preclinical pharmacology, animal intrathecal Cav3.2 antisense administration and genetic knockdown studies support the role for Cav3.2 channels in pain. Certain benzenesulfonamides are known to be T-type Cav3.2 calcium channel blockers. Specifically, they selectively blocks T-Type calcium channels. It would be particularly beneficial if such methods are based on previously unexplored mechanisms for pain treatment that may offer improved pain relief or are less associated with side effects. This invention provides compositions and methods of treatment that generally address such a need.

### SUMMARY

This invention is directed to methods for treating diabetic neuropathy, particularly diabetic peripheral neuropathy, in a subject in need of such treatment. The methods comprise administering to the subject an amount of therapeutic agent A. Therapeutic agent A is compound A or a salt thereof:

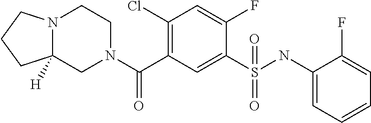 [SMILES: O=C(c1cc(S(=O)(=O)Nc2ccccc2F)c(F)cc1Cl)N1CCN2CCC[C@@H]2C1]

This invention also is directed to pharmaceutical compositions comprising an amount of therapeutic agent A. The compositions optionally can comprise one or more additional therapeutic agents.

This invention also is directed to a use of therapeutic agent A to prepare a medicament. The medicament optionally can comprise one or more additional therapeutic agents. In some embodiments, the medicament is useful for treating diabetic neuropathy, particularly, diabetic peripheral neuropathy.

This invention also is directed to methods of use of the above compositions, for example, to treat diabetic neuropathy, particularly diabetic peripheral neuropathy.

Further benefits of Applicants' invention will be apparent to one skilled in the art from reading this patent application.

### DETAILED DESCRIPTION OF THE INVENTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

As discussed above, this invention is directed, in part, to methods for treating diabetic neuropathy, such as, diabetic peripheral neuropathy, in a subject in need of such treatment. The methods comprise administering to the subject an amount of therapeutic agent A. Therapeutic agent A is compound A or a salt thereof. One example of a compound of Compound A is 4-chloro-2-fluoro-N-(2-fluorophenyl)-5-[(8aR)-hexahydropyrrolo[1,2-a]pyrazin-2(1H)-ylcarbonyl]benzenesulfonamide.

Compound A (including various salts thereof and crystalline forms of compound A and its salts) and compositions comprising compound A can be prepared as described in, for example, US Patent Application 2010/0197693, the contents of which are herein incorporated by reference.

As discussed above, this invention also is directed, in part, to pharmaceutical compositions comprising an amount of therapeutic agent A.

The compositions typically comprise one or more conventional pharmaceutically acceptable carriers, adjuvants, and/or vehicles (together referred to as “excipients”).

Compositions for oral administration, and solid dosage forms in particular, are preferred. Such solid dosage forms include, for example, capsules, tablets, pills, powders, and granules. In such solid dosage forms, the compounds or salts are ordinarily combined with one or more excipients. If administered per os, the compounds or salts can be mixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in, for example, a dispersion of the compound or salt in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can be prepared with enteric coatings.

The total daily dose of a Compound A or salt (administered in single or divided doses) is typically from about 1 to about 500 mg, more specifically, from about 5 to about 200 mg, and even more specifically, from about 10 to about 100 mg. Dosage unit compositions can contain such amounts or submultiples thereof to make up the daily dose. In many instances, the administration of the compound or salt will be repeated a plurality of times. Multiple doses per day typically may be used to increase the total daily dose, if desired.

Factors affecting the preferred dosage regimen include the type, age, weight, sex, diet, and condition of the patient; the severity of the pathological condition; the severity of the pathological condition; pharmacological considerations, such as the activity, efficacy, pharmacokinetic, and toxicology profiles of the particular compound or salt used; whether a drug delivery system is utilized; and the specific drug combination. Thus, the dosage regimen actually employed can vary widely, and therefore, can derive from the preferred dosage regimen set forth above.

This invention also is directed, in part, to a method for treating diabetic neuropathy in an animal in need of such treatment. More specifically, the method involves treating diabetic peripheral neuropathy in an animal in need of treatment thereof. These methods comprise administering to the animal a compound(s)/salt(s)/composition(s) of the invention. In some embodiments, a therapeutically effective amount of the compound/salt/combination/composition is administered to the animal. “Treating” means ameliorating, suppressing, eradicating, preventing, reducing the risk of, and/or delaying the onset of the disease being treated. Applicants specifically intend that the term “treating” encompass administration of the compositions of the invention to a patient suffering from diabetic neuropathy—such as diabetic peripheral neuropathy. The methods of treatment are particularly suitable for use with humans, but may be used with other animals, particularly mammals. A “therapeutically-effective amount” or “effective amount” is an amount that will achieve the goal of treating the targeted condition.

Peripheral neuropathy refers to a dysfunction of peripheral nerves the symptoms of which may include varying degrees of sensory disturbances, pain or muscular atrophy, diminished reflexes or vasomotor symptoms. Peripheral neuropathy includes any peripheral neuropathies induced by: toxin exposure (e.g. induced by exposure to e.g. pyridoxine, heavy metals, chemotherapeutic agents); myelin dysfunction (e.g., caused by immune response to viruses (e.g., HIV), bacteria (e.g., Campylobacter) or vaccines (e.g., influenza vaccine) or autoimmune response); compromised vascular supply to the peripheral nerves (e.g., chronic arteriosclerotic ischemia, vasculitis, hypercoagulable states); metabolic states (e.g., diabetes mellitus, chronic renal insufficiency,); trauma (e.g., muscular overextension, pressure, compression, hemorrhage, exposure to cold or radiation, direct tumor invasion). As mentioned previously herein, in certain aspects, the methods of the present invention relate to treating patients suffering from diabetic peripheral neuropathy. One technique that can be used to assess peripheral diabetic neuropathy in humans is using microneurography, which is a technique well known in the art. Microneurography is a technique in which the activity of single nerve fibers can be studied and it is the only technique for directly assessing the function of peripheral nerves in pathological states in humans. Microneurography can provide valuable information of proof of mechanism for investigational compounds. Microneurography is considered safe and minimally invasive.

This invention also is directed, in part, to a use of the compound(s)/salt(s)/composition(s) of the invention, and, optionally one or more additional therapeutic agents to prepare a medicament.

This invention also is directed, in part, to a use of therapeutic agent A and, optionally one or more additional therapeutic agents to prepare a medicament.

In some embodiments, the above medicaments are for co-administration with one or more additional therapeutic agents.

In some embodiments, the medicaments are for treating diabetic neuropathy. In another embodiment, the medicaments are for treating diabetic peripheral neuropathy.

This invention also is directed, in part, to one or more compound(s)/salt(s)/composition(s) of the invention, and, optionally one or more additional therapeutic agents, for use as a medicament.

This invention also is directed, in part, to combination of therapeutic agent A and, optionally one or more additional therapeutic agents, for use as a medicament.

### EXAMPLE

The following example is merely illustrative, and not limiting to this disclosure in any way.

### Example 1

### A Randomized, Double-Blind, Placebo- and Active-Controlled Study of the Electrophysiological Effects of Compound A on Spontaneous Activity in C-Nociceptors in Patients with Diabetic Peripheral Neuropathy

The following list of abbreviations are used in this Example 1:

### AE Adverse Event

ALT Alanine transaminase
AST Aspartate transaminase

### A-V Atrioventricular

### BID Twice Daily

### BMI Body Mass Index

BP Blood pressure
BPM Beats per minute

### CRF Case Report Form

### CYP3A Cytochrome P450 3A

### DPN Diabetic Peripheral Neuropathy

### ECG Electrocardiogram

EDTA Edetic acid (ethylenediaminetetraacetic acid)

### FDA U.S. Food and Drug Administration

FIH First in human

### GCP Good Clinical Practice

### GDRD Global Pharmaceutical Research and Development

HBsAg Hepatitis B surface antigen
HCV Ab Hepatitis C virus antibody

### HIV Human Immunodeficiency Virus

### ICH International Conference on Harmonization

### IEC Independent Ethics Committee

### IMP Investigational Medicinal Product

### IRB Institutional Review Board

IUD Intrauterine device
IUS Intrauterine system

### IV Intraveneous

### MedDRA Medical Dictionary for Regulatory Activities

N Total spikes
NSAID Nonsteroidal anti-inflammatory drug

### NRS Numerical Rating Scale

QTc QT interval corrected for heart rate
QTcF QT interval corrected for heart rate, Fredericia method

### RPL Richmond Pharmacology Limited

### SAE Serious Adverse Event

### SLI Significant Latency Increase

### SOC System Organ Class

### TI Total Increase

### Pharmacokinetic and Statistical Abbreviations

ANOVA Analysis of variance
AUC Area under the plasma concentration-time curve
AUCₜ Area under the plasma concentration-time curve from time zero to time of last measurable concentration
Cₘₐₓ Maximum observed plasma concentration
MΩ Megaohm
Tₘₐₓ Time to maximum observed plasma concentration
mm Micrometer

The objective of this study is to assess the effect of a single dose of Compound A and intravenous lidocaine 3 mg/kg on spontaneous activity in peripheral C-nociceptors measured through microneurography in subjects with painful diabetic peripheral neuropathy (DPN) compared to placebo. The safety, tolerability, pharmacodynamic and pharmacokinetics of a single dose of Compound A will also be assessed.

### 1.0 Investigational Plan

### 1.1 Overall Study Design and Plan: Description

This is a Phase 2, single-dose, double-blind, parallel group and randomized study designed to assess the electrophysiological effects of Compound A on spontaneous activity in C-nociceptors in subjects with painful diabetic peripheral neuropathy. Adult male and female subjects with diabetic neuropathic pain will be selected to participate in the study according to the selection criteria.

The study is designed to enroll up to 48 subjects in whom at least one C-fiber can be identified with microneurographic recordings for a minimum of 1 hour post oral dosing to meet scientific and regulatory objectives without enrolling an undue number of subjects in alignment with ethical considerations. Therefore, if the target number of subjects has been enrolled, there is a possibility that additional subjects in screening will not be enrolled.

### Screening/Washout Period

Each subject will have a Screening Visit within 30 days of Day 1. The Screening Period will consist of 2 visits (Visit 1 and Visit 2) as outlined in Table 1. The screening procedures specified in Table 1 must be conducted. There must be a washout of adequate length for all the types of medications specified in Section 1.1.3. The washout must be at least 5 half-lives of the longest acting prohibited medication or 2 days, whichever is longer.

Paracetamol up to 3000 mg/day is permitted as a rescue medication during Screening/Washout Period for subjects required to wash out of prohibited analgesic medications. Subjects who are allowed to continue their prescribed analgesic will not be allowed to use paracetamol as rescue medication. Paracetamol as a rescue medication will not be allowed on Day 1.

Subjects will complete assessments of pain intensity associated with diabetic neuropathy on a daily basis on a paper diary using an 11-point (0-10) numerical rating scale (NRS) for approximately 7 consecutive (minimum of 5) days. The subjects are required to have an average daily pain score of at least 4 on NRS prior to Day −1 and to have a score of at least 4 on Day −1 to be eligible.

### Treatment Period

Following the screening period, eligible subjects will return on Day −1. The subjects will be confined overnight on Day −1. On Day 1 microneurography will be performed. Approximately 10 to 15 minutes of baseline activity of C-nociceptors will be recorded. Subjects who have at least one C-fiber with spontaneous C-nociceptor activity identified will be randomized into the study and assigned in an approximate 2:1:1 ratio to receive a single dose of Compound A, placebo, or lidocaine as listed below. Measures of spontaneous activity via microneurography will be made prior to, during and after drug administration. The investigator and the study subjects will remain blinded to the treatment throughout the study.

The three regimens that will be studied are as follows:
Regimen A 100 mg Compound A (two 50 mg capsules, oral), IV placebo (glucose)
Regimen B 3 mg/kg Lidocaine IV infused over 30 minutes, 2 placebo capsules for Compound A (oral)
Regimen C Placebo [2 placebo capsules for Compound A (oral), placebo IV (glucose)]

Subjects will receive an oral dose and an IV dose of study medication on Study Day 1 under fasting conditions. Each oral dose of study drug will be taken orally with enough water to swallow the study medication. The oral dose of study medication will be administered at 0 hour. The IV dose of study medication will be administered 30 minutes following the oral dose at 0.5 hour. The IV dose will be infused over 30 minutes.

On Day 1 the intensity of ongoing pain related to diabetic neuropathy will be assessed verbally using NRS by the subject and will be collected at baseline (immediately prior to microneurography) and every hour following the oral study drug administration until the microneurography procedure is complete.

Subjects will be confined on Study Day −1 until after the collection of the 4 hour blood samples and scheduled study procedures on Study Day 1. Serial blood samples will be collected for 4 hours after the oral dose. Safety will be assessed throughout the study.

A follow-up phone call (or contact) will be conducted approximately 3 days after dosing for all subjects.

### 1.1 Selection of Study Population

Subjects will undergo screening procedures within 30 days prior to initial study drug administration. Adult male and female subjects with diabetic neuropathic pain who meet the inclusion criteria and who do not meet any of the exclusion criteria will be eligible for enrollment into the study.

### 1.1.1 Inclusion Criteria

A subject will be eligible for study participation if he/she meets the following criteria:
1. Male or female between 18 and 75 years of age, inclusive, at Screening Visit.
2. If female, subject of childbearing potential must agree to use medically acceptable methods of contraception from the time of signing the informed consent until 3 months following administration of the last treatment or dose of study medication as outlined below:
   A documented placement of an intrauterine device (IUD) or intrauterine system (IUS) and the use of a barrier method {condom or occlusive cap (diaphragm or cervical/vault caps) used with spermicidal foam/gel/film/cream/suppository};
   Oral contraceptives (combination oestrogen/progesterone pills), injectable progesterone or subdermal implants and the use of a barrier method {condom or occlusive cap (diaphragm or cervical/vault caps) used with spermicidal foam/gel/film/cream/suppository}
   Documented tubal ligation (female sterilization). In addition, a barrier method {condom or occlusive cap (diaphragm or cervical/vault caps) used with spermicidial foam/gel/film/cream/suppository} should also be used;
   Double barrier method: Condom and occlusive cap (diaphragm or cervical/vault caps) with spermicidal foam/gel/film/cream/suppository;
   True abstinence: When this is in line with the preferred and usual lifestyle of the subject. [Periodic abstinence (e.g., calendar, ovulation, symptothermal, post-ovulation methods) and withdrawal are not acceptable methods of contraception].
3. Females must not have a positive results for pregnancy tests performed:
   At Screening on a serum sample obtained within 30 days prior to Day 1;
   On a serum sample obtained on Day −1.
4. Male subjects must utilize at least one of the following:
   use a condom with spermicidal foam/gel/film/cream/suppository if their female partner(s) is (are) pregnant or lactating from the time of the first administration of treatment or study medication until 3 months following administration of the last treatment or dose of study medication.
   Use acceptable methods of contraception if the male subject's partner could become pregnant from the time of the first administration of treatment or study medication until 3 months following administration of the last treatment or dose of study medication. The acceptable methods of contraception are as follows:
      Condom and occlusive cap (diaphragm or cervical/vault caps) with spermicidal foam/gel/film/cream/suppository;
      Surgical sterilisation (vasectomy with documentation of azoospermia) and a barrier method {condom or occlusive cap (diaphragm or cervical/vault caps) used with spermicidal foam/gel/film/cream/suppository};
      The female partner uses oral contraceptives (combination oestrogen/progesterone pills), injectable progesterone or subdermal implants and a barrier method {condom or occlusive cap (diaphragm or cervical/vault caps) used with spermicidal foam/gel/film/cream/suppository};
      The female partner uses medically prescribed topically-applied transdermal contraceptive patch and a barrier method {condom or occlusive cap (diaphragm or cervical/vault caps) used with spermicidal foam/gel/film/cream/suppository};
      The female partner has undergone documented tubal ligation (female sterilisation). In addition, a barrier method {condom or occlusive cap (diaphragm or cervical/vault caps) used with spermicidal foam/gel/film/cream/suppository} must be used;
      The female partner has undergone documented placement of an IUD or IUS and the use of a barrier method {condom or occlusive cap (diaphragm or cervical/vault caps) used with spermicidal foam/gel/film/cream/suppository};
      True abstinence: When this is in line with the preferred and usual lifestyle of the subject. [Periodic abstinence (e.g., calendar, ovulation, symptothermal, post-ovulation methods) and withdrawal are not acceptable methods of contraception].
5. Body Mass Index (BMI) at Screening is 18.5 to 33, inclusive. BMI is calculated as weight in kg divided by the square of height measured in meters.
6. Subject has a diagnosis of diabetes mellitus type 1 or type 2.
7. Subject must have clinical evidence of diabetes-related peripheral neuropathy in the distal lower extremities and presence of pain due to diabetic peripheral neuropathy in the distal lower extremities for at least 6 months prior to study entry.
8. Subject has a stable diabetic treatment regimen, including oral hypoglycemics, insulin, or diet for 3 months before Screening.
9. Subject is willing to discontinue prohibited medications.
10. Subject must have an average daily pain score of (greater than or equal to)≧4 on an 11-point (0-10) numerical rating scale collected via paper diary over 7 consecutive days (minimum 5 days) prior to the Day −1 visit and on Day −1.
11. Hemoglobin A_{1c} level ≦11%.
12. Subject must voluntarily sign and date an informed consent form, approved by an Independent Ethics Committee (IEC)/Institutional Review Board (IRB), prior to the conduct of any study-specific procedures.

### 1.1.2 Exclusion Criteria

A subject will not be eligible for study participation if he/she meets any of the following criteria:
1. If female, subject is pregnant or breast feeding, or plans to become pregnant or breast feed during the course of the study.
2. History of significant sensitivity to any drug, particularly any allergic reaction or hypersensitivity to lidocaine or similar medications.
3. Dermatologic or vascular disease in the feet that may interfere with assessment, including diabetic ulcer or any toe or limb amputation.
4. Currently receiving warfarin or heparin.
5. Hospitalizations within the past 1 month for episodes of hypoglycemia or hyperglycemia.
6. Subject has positive screening laboratory results for Human Immunodeficiency Virus (HIV), Hepatitis B surface antigen (HBs Ag) or Hepatitis C antibody (HCV Ab).
7. Subject has a history of drug (licit or illicit) or alcohol abuse/addiction within the last 6 months.
8. Subject has positive findings from the drug screen with the exception of positive results for a known prescribed medication.
9. Subject has a history of head trauma with loss of consciousness, history of epilepsy, seizures or convulsions, including febrile, alcohol or drug withdrawal seizures.
10. Use of any known strong CYP3A4 inhibitors (e.g., ketoconazole) or dual CYP3A4 and 2C9 inhibitor (e.g., fluconazole), or dual CYP3A4 and 2C9 inducer (e.g., rifampin) within 1 month prior to study drug administration.
11. Subject has a history of gastric surgery, vagotomy, bowel resection or any surgical procedure that might interfere with gastrointestinal motility, pH or absorption.
12. Subject has clinically abnormal laboratory value, at Screening and/or Day −1 in the opinion of the investigator.
13. History of cardiovascular abnormality, including left ventricular dysfunction, sick sinus syndrome, family history of long-QT syndrome, or unexplained sudden deaths in their family.
14. Has a clinically significant abnormal ECG or an ECG with a QTc interval corrected for heart rate using the Fridericia formula (QTcF)>450 msec for men and >470 msec for women with normal QRS duration or second degree A-V block Type II, third degree A-V block, atrial flutter, atrial fibrillation or an accessory bypass track (e.g., Wolff-Parkinson-White syndrome) at Screening or Day 1.
15. Subject has any of the following:
   Hypertension with a systolic blood pressure (BP)≧160 mmHg or a diastolic BP≧100 mmHg at Screening or Day −1.
   Hypotension with a systolic BP≦90 mmHg or a diastolic BP≦60 mmHg at Screening or Day −1.
   A heart rate ≧100 or <45 beats per minute (BPM) at Screening or Day −1.
16. Subject has previously participated in this study or any other Compound A study or current participation in another clinical study.
17. Subject is considered by the investigator, for any reason, to be an unsuitable candidate to receive Compound A, lidocaine, or microneurography procedure.
18. Received an investigational drug within 90 days or used an investigational device within 60 days before planned start of treatment, topical capsaicin within 6 months or a lidocaine patch within 30 days prior to Day 1.
19. Employees of the investigator center, with direct involvement in the proposed study or other studies under the direction of that investigator or centre, as well as family members of the employees or the investigator.
20. Subject in the opinion of the investigator has any clinically significant infection/illness/injury.
21. Subject has an active malignancy of any type or has been diagnosed or treated for cancer within the past 5 years. Subjects with basal cell carcinoma of the skin that has been successfully treated will be allowed to participate.
22. Any uncontrolled clinically significant cardiac, respiratory (except mild asthma), renal, hepatic, gastrointestinal, hematologic, endocrine, dermatological, metabolic, neurologic (nerve injury) or psychiatric disease or disorder, or any uncontrolled medical illness.
23. Presence of edema or any skin condition at the ankle level that may interfere with the microneurography procedure.

### 1.1.3 Prior and Concomitant Therapy

Any medication, vaccines and over-the-counter medicines, vitamins and/or herbal supplements, etc. that the subject has taken within thirty (30) days prior to randomization must be recorded, along with the reason for use, dates of administration including start and end date, dosages and frequency.

Subjects must discontinue the following medications (including but not limited to these medications) during the Screening/Washout Period:
topical analgesics containing lidocaine or capsaicin,
sodium channel blockers (lamotrigine, lacosamide, valproate, quinidine, procainamide, lidocaine, mexiletine and tocamide),
potassium blockers (sotalol and amiodarone),
calcium channel blockers (diltiazem and verapamil, levetiracetam),
strong CYP3A4 inhibitor (ketoconazole), dual CYP3A4 and 2C9 inhibitor (fluconazole) and dual CYP3A4 and 2C9 inducer (rifampin)

The length of the medication washout will be based upon the half-life of the longest acting agent being used and must equal a duration of at least 5 drug half-lives of the longest acting agent or 2 days, whichever is longer. In other words, if a subject is taking more than one prohibited agent at the time of the Screening Visit, the duration of the washout is determined by the agent with the longest acting half-life. The appropriateness of washout of prohibited medication should be carefully evaluated by the investigator to assess participation in the study.

On Day 1, all medications mentioned above will not allowed before the completion of microneurography.

Topical medications including topical corticosteroids are not allowed on Day −1 and Day 1.

Paracetamol and paracetamol-containing products may be used to treat conditions other than diabetic neuropathic pain during Screening/Washout Period up to Day −1. Subjects should not take more than a total of 3000 mg of paracetamol per day. The use of these products for non-diabetic neuropathic pain conditions will be captured as a concomitant medication in the case report form (CRF).

In addition to the prohibited medications mentioned in this protocol, the Investigator should refer to the lidocaine product label and review a subject's entire concomitant therapy use to ensure each therapy (both single and combination agents) is appropriate for the subject. The Investigator should refer to the lidocaine product label for other potential safety-related events such as dizziness, nervousness, tremor, hypotension and bradycardia.

Treatment of study drug side effects is permissible with an agent of the Investigator's choice provided it is not a prohibited concomitant medication. If a prohibited medication is necessary to treat an adverse event the Study Designated Physician should be consulted.

Subjects should continue all other medications for diabetes, DNP and other medical conditions as long as these medications are not prohibited as listed above. Over-the-counter medications, vitamins and herbal supplements are allowed unless otherwise prohibited.

### Rescue Therapy

Paracetamol up to 3000 mg/day is permitted as a rescue medication during Screening/Washout Period only for those subjects who are required to wash out of a prohibited analgesic medication. Subjects who are allowed to continue on their prescribed analgesic medication will not be allowed to use paracetamol during screening or wash out phase. Paracetamol is not allowed on Day 1 before the completion of microneurography procedure for any subjects. Subjects should not take more than a total of 3000 mg per day of paracetamol for rescue. If a subject takes paracetamol or paracetamol containing products for other conditions, the combined total daily dose should not exceed 3000 mg. Paracetamol or paracetamol containing products used for purposes other than rescue medication will be recorded as a concomitant medication in the CRF.

### 2.0 Efficacy, Pharmacokinetic, Pharmacodynamic, and Safety Assessments/Variables

### 2.1 Efficacy and Safety Measurements Assessed and Flow Chart

This study is not designed to assess efficacy but to assess pharmacodynamic effect. Study procedures described in this protocol are summarized in Table 1.

TABLE 1
Study Activities

| | | | | | Prior to Discharge or | |
|---|---|---|---|---|---|---|
| | Screening^{a} | | | | Premature | Follow-up |
| Activity | Visit 1 | Visit 2 | Day −1 | Day 1 | Discontinuation | Contact^{b} |
| Informed Consent^{c} | X | | | | | |
| Medical History | X | X^{d} | X^{d} | | | |
| Physical Examination^{e,f} | X | | X | | X | |
| Total Neuropathic Examination^{g} | X | | X | | X | |
| 12-Lead ECG^{h} | X | | X | | X | |
| 24 hour Holter Monitoringⁱ | X | | | | | |
| Continuous Telemetry Monitoring^{j} | | | | X | | |
| Vital Signs^{k} | X | | X | X | X | |
| Clinical Laboratory Tests^{l} | | X | X | | X | |
| Glycemic Levels^{m} | | | | X | X | |
| HBsAg, HCV Ab, Anti-HIV ab Tests | | X | | | | |
| Urine Drug/Alcohol Screen | X | | X | | | |
| Pregnancy Test | | X | X | | X | |
| Study Drug Administration | | | | X | | |
| Blood Samples for Drug Assayⁿ | | | | X | | |
| Microneurography | | | | X | | |
| Diary | X | | | | | |
| Neuropathic Pain Assessment (NRS)^{o} | | X | X | X | | |
| Other Medication Assessment | X | X | X | X | | |
| Adverse Event Assessment^{p} | | | | X | X | X |

^{a}Perform within 30 days prior to initial study drug administration (Day 1).
^{b}Follow up contact will be made approximately 3 days after discharge.
^{c}Prior to performing any screening or study-specific procedures.
^{d}Medical history update.
^{e}Weight included at Screening and on Day −1. Physical exam conducted at Screening, Day −1, and prior to discharge from the site.
^{f}Height measured at Screening only.
^{g}Neuropathic exam will be performed at Screening, Day −1, and prior discharge or upon premature discontinuation.
^{h}ECGs will be performed at Screening, on Day −1 and prior to discharge or upon premature discontinuation.
ⁱSubjects will have a Holter monitor for 24 hours during the Screening Period.
^{j}Continuous telemetry monitoring beginning approximately 30 minutes prior to the oral dose and continuing through the completion of the 4 hour post oral dose assessments.
^{k}Vital signs will be measured at Screening, on Day −1 and prior to oral dosing on Day 1, at 2 hours post oral dosing on Day 1 and prior to discharge or upon premature discontinuation. Body temperature will be measured at Screening, on Day −1, and prior to oral dosing on Day 1 and prior to discharge or upon premature discontinuation.
^{l}Clinical laboratory tests including urinalysis will be conducted at Screening, on Day −1 (if the Screening visit is not within 14 days of Day 1), and prior to discharge, and at premature discontinuation.
^{m}Glycemic levels will be obtained prior to oral dosing (0 hour) and at 0.50, 1, 1.5, 2, 2.5, 3.5 and 4 hours after oral dosing (prior to discharge) or upon premature discontinuation.
ⁿBlood samples for Compound A and lidocaine assays will be collected at 0.50, 0.75, 1, 1.5, 2, 3 and 4 hours after oral dosing.
^{o}Neuropathic pain assessment via an 11-point numeric scale will be assessed for 7 days prior to the Day −1 visit, on Study Day −1, prior to oral dose on Day 1 and every hour during the microneurography assessment.
^{p}A detailed description for procedures involving adverse event assessments can be found in Section 0.

### 2.2 Study Procedures

### Medical History

A complete medical history, including alcohol, tobacco and nicotine-containing product use histories, will be taken at Screening. The medical history will be updated at the Screening Visit 2 and the Study Day −1. The medical history obtained at Screening Visit 2 will serve as the baseline for clinical assessment.

Medication (prescription or over-the-counter, including vitamins and herbal supplements) use from 30 days prior to study drug administration through the end of the study will also be recorded.

### Hepatitis Screen

HBsAg and HCV Ab tests will be performed during Screening. The hepatitis test panel will be performed by a certified laboratory.

### HIV Screen

Subjects will have blood tested by a certified laboratory for the presence of anti-HIV Ab during Screening. Only those subjects negative for the presence of antibodies will be allowed to enroll in the study. The results of the HIV Ab testing will be retained by the study site under confidential restriction.

### Physical Examination

A physical examination will be performed at Screening, upon check in on Study Day −1, and prior to discharge from the site or upon premature discontinuation. A symptom-directed physical examination will be performed when necessary. Height will be measured only at Screening; the subject will not wear shoes. The physical examination performed at Screening will serve as the baseline physical examination for clinical assessment. Any significant physical examination findings after dosing will be recorded as adverse events.

Body weight will be measured at Screening (baseline for clinical assessment) and upon check in on Study Day −1. The subject will wear lightweight clothing and no shoes during weighing.

### Total Neuropathic Exam

The total neuropathic exam will include the following assessments:
Sensory symptoms
Motor symptoms
Autoimmune symptoms
Pin sensibility
Vibration sensibility
Strength
Deep tendon reflexes

The total neuropathy exam will be performed at Screening, Day −1, prior to discharge and upon premature discontinuation.

### Vital Signs

Semi-recumbent blood pressure (BP) and pulse rate will be measured using a semi-automatic BP recording device (Dinamap® monitors) with an appropriate cuff size. BP and pulse rate will be measured after the subject has been resting in a semi-recumbent position for at least 5 minutes and prior to any scheduled blood draws. For timings of individual measurements refer to the Study Activities (Table 1).

The vital signs measurements on Study Day 1 prior to the oral dose will serve as the baseline measurements for clinical assessment.

### Body Temperature

Body temperature (tympanic) will be measured (a single measurement) in degrees Celsius using an automated thermometer at the times indicated in the Study Activities (Table 1). Additional temperature assessments may be taken for safety at the discretion of the Principal Investigator or delegate.

The body temperature measurement prior to oral dosing on Study Day 1 will serve as the baseline measurements for clinical assessment.

### Electrocardiograms

### 12-Lead Electrocardiogram (ECG)

A 12-lead resting ECG will be obtained as outlined in Table 1.

The 12-lead ECG measurements on Day −1 will serve as the baseline measurements for clinical assessment.

The assessments of the 12-lead ECGs will be recorded in the case report forms (CRFs). The original ECG tracing will be retained in the subject's records at the study site. An appropriately qualified physician (preferably a cardiologist) at the study site (local reader) will read all ECGs. The local reader will record his/her global interpretation as either “normal ECG,” “abnormal ECG—not clinically significant” or “abnormal ECG—clinically significant,” directly on the ECG CRF pages. Only the local reader's reading of the ECG will be collected. The ECG CRF page will be considered the source document for these ECG assessments. The automatic machine reading (i.e., machine-generated measurements and interpretation that are automatically printed on the ECG tracing) will not be collected. The QT interval measurement (corrected by Fredericia formula, QTcF) will be documented only if the local reader selects the “prolonged QT” box.

### Screening Hotter Recordings

A 5-lead Holter recording will be performed to store continuous ECGs. During the Screening Period, the subjects will be on a Holter monitor for 24 hours. The screening Holter reports will be reviewed and signed off by a qualified cardiologist to assess the subject's eligibility for the trial.

### Continuous 12 Lead Telemetry

Subjects will be monitored by telemetry from approximately 30 minutes prior to the oral dose until the 4 hours post dosing. Telemetry will be monitored using a Surveyor™ 12-lead system (Mortara Instrument Inc.). The system will be managed according to local working practices. This assessment is used only for safety monitoring. Data will be downloaded and stored electronically.

### Urine Screens for Drugs of Abuse and Alcohol

### Urine Screens for Drugs of Abuse

Urine will be tested for the following drugs of abuse: benzodiazepines, opiates to include oxycodone, amphetamines, methadone, cocaine, cannabinoids, and barbiturates at RPL (for details see Table 1). A repeat drug screen can be done where methodological reasons are believed to have led to a false positive in line with RPL's SOPs. Borderline positive results, unless covered by the preceding condition, are to be considered as positive and the subject excluded from the study. If subjects are found to be positive due to medication e.g., flu/cold remedies they may undergo a repeat drug screen if they are still within the screening window.

### Alcohol Breath Test

An alcohol breath test will be done (for details see Table 1), using an alcometer.

### Pregnancy Tests

To exclude pregnancy, a serum pregnancy test will be performed at screening and on admission (Day −1) (for details see Table 1). A urine and/or serum pregnancy test will be performed whenever pregnancy is suspected. Any subject with a positive pregnancy test will be excluded or withdrawn.

These analyses will be performed by the certified laboratory chosen for the study.

### Clinical Laboratory Tests

Samples will be obtained at a minimum for the clinical laboratory tests outlined in Table 2. Samples will be obtained during Screening, upon study check in on Day −1 (if the Screening visit is not within 14 days of Day 1), upon subject premature discontinuation and prior to discharge.

Additional glycemic levels will be obtained prior to dosing (0 hour) and at 0.5, 1, 1.5, 2, 2.5, 3.5 and 4 hours after dosing on Study Day 1, or upon subject discontinuation.

Urine samples for determination of urinalysis parameters will be taken at the times given in the study activities (Table 1). Urinalysis will be performed by RPL using a dipstick method, which provides information regarding leukocytes, nitrite, urobilinogen, protein, pH, blood, specific gravity, ketones, bilirubin, and glucose in/of the urine. If deemed necessary, based on a clinically significant positive test, microscopic examination of sediment and/or culture will be performed by The Doctors Laboratory.

A certified laboratory will be utilized to process and provide results for the clinical laboratory tests. The baseline laboratory test results for clinical assessment for a particular test will be defined as the last measurement prior to the initial dose of study drug. The certified laboratory chosen for this study is:

The Doctor's Laboratory

60 Whitfield Street

London

W1T 4EU

TABLE 2
Clinical Laboratory Tests

Hematology
Hematocrit
Hemoglobin
Red blood cell (RBC) count
White blood cell (WBC)
count
Neutrophils
Bands (if detected)
Lymphocytes
Monocytes
Basophils (if detected)
Eosinophils (if detected)
Absolute platelet count
Coagulation
Prothrombin Time (PT)
Activated partial
thromboplastin
time (aPTT)
Clinical Chemistry
Blood urea nitrogen (BUN)
Creatinine
Direct bilirubin
Total bilirubin
Serum glutamic-pyruvic
transaminase
(SGPT/ALT)
Serum glutamic-oxaloacetic
transaminase
(SGOT/AST)
Alkaline phosphatase
Sodium
Potassium
Chloride
Calcium
Inorganic phosphorus
Uric acid
Cholesterol
Total protein
Glucose*
Triglycerides
Albumin
Urinalysis
Specific gravity
Ketones
pH
Protein
Blood
Glucose
leukocytes
nitrites
urobilinogen
bilirubin
Microscopic exam (if
clinically indicated)
*Glycemic levels will be obtained prior to dosing (0 hour) and at 0.50, 1, 1.5, 2, 2.5, 3.5 and 4 hours after dosing on Study Day 1, or upon subject discontinuation.

For any laboratory test value outside the reference range that the investigator considers to be clinically significant:
The investigator will repeat the test to verify the out-of-range value.
The investigator will follow the out-of-range value to a satisfactory clinical resolution.

TABLE 3
Blood Sampling Volumes

| | Maximum Number | ML of Blood | Total Volume |
|---|---|---|---|
| Assessment | of Samples | per Sample | of Blood |
| Hematology | 4 | 4 ML | 16 ML |
| Clinical Chemistry | 4 | 5 ML | 20 ML |
| Coagulation | 4 | 4.5 ML | 18 ML |
| Pharmacokinetic | 7 | 4 ML | 28 ML |
| | | Total | 82 ML |

### Microneurographic Recordings

Microneurography will be used to record C-fiber action potentials from the superficial peroneal nerve at the ankle, using tungsten microelectrodes (200 μm diameter, lacquer-insulated, nominal impedance 1MΩ). A subcutaneous reference electrode will be inserted in the skin approximately 2 cm outside the nerve trunk. Temperature of the skin will be measured with a thermocouple placed on the skin adjacent to the receptive fields of the units under study. Electrical stimuli will be triggered, and the responses to electrical stimulation digitized with a data acquisition board and recorded in a computer. Digital filtering (band pass 0.3-2 kHz) and clamping of the baseline will be performed both on-line and during off-line analysis for a better visualization of the action potentials.

On Day 1, for approximately 10 to 15 minutes, baseline activity of C-fibers will be recorded prior to oral dosing. The recording will continue following oral dosing up to 3 hours maximum.

### Pain Intensity Measure

At the Screening Visit 1, the subjects will be provided a paper diary and will be required to complete the diary every evening for about 7 days (minimum 5 days) prior to Day −1. Subjects will record their assessment of pain associated with diabetic neuropathy on an 11-point numerical rating scale. Beginning on Day 1 subjects will verbally rate their pain using a NRS immediately prior to oral dosing and every hour until completion of the microneurography assessments or upon premature discontinuation.

### Randomization and Assignment of Subject Numbers

The results of all screening and prior to dose Study Day 1 evaluations must be within clinically acceptable limits, upon review by the investigator, before a subject can be administered study drug. Subjects who meet the inclusion criteria and do not meet any of the exclusion criteria will proceed to randomization. In addition, only subjects who have spontaneous C-nociceptor activity identified will be randomized into the study.

As they are enrolled into the study, subjects will be assigned unique consecutive numbers and randomized to one of the regimens shown in Section 4.5.

### 2.3 Confinement

Subjects will be confined to the study site and supervised from Day −1 to Day 1. Confinement in each period will begin on Study Day −1 until after the collection of the 4-hour blood samples and scheduled study procedures are completed on Study Day 1. Strenuous activity, smoking and alcohol consumption during confinement will not be permitted.

### 2.4 Meals and Dietary Requirements

Subjects will receive a standardized diet for all meals during confinement. Subjects can only consume water to quench thirst within 8 hours prior to oral dosing. A light breakfast will be provided to the subject who is to receive the microneurography procedure in the afternoon such that they will have eaten at least 8 hours prior to oral study drug administration. During the microneurography procedure subjects that have a low glucose level may have a light high carbohydrate snack or sugar drink.

Subjects will not consume alcohol or caffeine during confinement and within 24 hours prior to study drug administration.

### 2.5 Drug Concentration Measurements

Collection of Samples for Analysis

Blood samples for Compound A and lidocaine assays will be collected by an indwelling intravenous catheter or venipuncture at 0.50, 0.75, 1, 1.5, 2, 3 and 4 hours after dosing. Samples will be taken within ±5 minutes of the scheduled times. If an indwelling catheter is used for blood collection, then 3 catheter volumes of blood must be collected and discarded prior to sample collection. The catheter should not contain silicone.

The order of blood collections will be maintained to the minute such that the time intervals relative to the preceding dose will be the same for all subjects. The time that each blood sample is collected will be recorded to the minute.

The blood samples will be collected in 4 mL evacuated potassium K2 EDTA-containing collection tubes. Sufficient blood will be collected to provide approximately 2 mL plasma from each sample Immediately after collection, the blood samples will be inverted several times to ensure good mixing of the blood and anticoagulant, and will be placed in an ice bath.

A total of 7 blood samples are planned to be collected per subject for pharmacokinetic analysis.

### 2.6 Handling/Processing of Samples

The blood samples for Compound A and lidocaine will be centrifuged within 30 minutes of collection using a refrigerated centrifuge at sufficient force to separate the plasma for approximately 10 minutes. The plasma samples will be transferred equally using plastic pipettes into two screw-capped polypropylene tubes clearly marked as “PK Compound A” and “PK Lidocaine” labeled with the drug number, type of sample (plasma), the protocol number, the subject number, the study period and study day, and the planned time of sampling relative to oral dosing. The plasma samples will be frozen within 1 hour after collection and maintained at −20° C. or colder until shipped/transferred to study sponsor.

### 2.7 Disposition of Samples

The frozen plasma samples for Compound A and lidocaine will be packed in dry ice sufficient to last during transport and shipped/transferred from the study site to study sponsor according to instructions from study sponsor. An inventory of the samples included will accompany the package. Arrangements will be made with study sponsor for the shipment/transfer of samples.

On the day of transfer, a copy of the inventory sheet will be faxed/e-mailed to the Sample Receiving Department.

### 2.8 Measurement Methods

### Analysis of Plasma Samples

Plasma concentrations of Compound A will be determined under the supervision of the Drug Analysis Department at study sponsor using a laser diode thermal desorption (LDTD)/mass spectrometry method. Evaluation of possible metabolites of Compound A will be determined using validated or non-validated methods. Plasma concentrations of lidocaine will be determined under the supervision of the Drug Analysis Department at study sponsor using a validated liquid chromatography/mass spectrometry method.

### 2.9 Efficacy Variables

### Please see Section 3.1.

### 3.0 Safety Variables

The following safety evaluations will be performed during the study: adverse event monitoring and vital signs, physical examination, ECG and laboratory tests assessments.

### 3.1 Pharmacokinetic Variables

Values for the pharmacokinetic parameters of Compound A and lidocaine, including the maximum observed plasma concentration (Cₘₐₓ), the time to Cₘₐₓ (peak time, Tₘₐₓ), and the area under the plasma concentration-time curve (AUCₜ) from time 0 to the time of the last measurable concentration (AUCₛ), will be determined using noncompartmental methods. Additional parameters may be calculated if useful in the interpretation of the data. In particular, pharmacokinetic parameters of metabolites of Compound A may be calculated if sufficient concentrations are found.

### 3.2 Pharmacodynamic Variables

The following will be determined for 10-minute intervals after the oral dose for each C-fiber for which microneurography recording is done. Note that recording may be done for a subject on more than one C-fiber.
Number of Significant Latency Increases (SLI) (any departure >300 μs from baseline latency) per minute.
Maximum Increase: the largest individual SLI expressed as a percentage of baseline latency.
Total Increase (TI): sum of individual SLI increases, with an increase measured as percentage of baseline latency. For a given time interval, TI is greatly influenced by the number of extra spikes that occur in the time interval.
Total spikes (N): calculated number of extra spikes derived from ΣSLI in a given unit of time, adjusted to the individual fiber percentage latency change for 2 extra spikes at the beginning of the 2 Hz period.
Spikes/min: calculated number of extra spikes per minute.
   3.3 Removal of Subjects from Therapy or Assessment

### 3.4 Discontinuation of Individual Subjects

Each subject has the right to withdraw from the study at any time. In addition, the investigator may discontinue a subject from the study at any time if the investigator considers it necessary for any reason, including the occurrence of an adverse event or noncompliance with the protocol. Subjects who discontinue the study due to the microneurographic reading being lost or unable to hold the position for microneurographic reading before oral dosing or within 1 hour following dosing will be replaced. Subjects who withdraw from the study may be replaced.

In the event that a subject withdraws or is discontinued from the study, the reason(s) for the discontinuation from the study will be recorded and a physical examination, body weight, vital signs measurement, ECG, laboratory analyses (including pregnancy test if applicable), total neuropathic exam, pain intensity assessments and an assessment of adverse events will be performed as soon as possible after discontinuation from the study. Additional blood samples for drug measurement may be collected at the time of discontinuation from subjects who are discontinued due to adverse events; the clock time, and date the sample was taken will be recorded.

If a subject is discontinued from the study with an ongoing adverse event or an unresolved laboratory result that is significantly outside of the reference range, the investigator will attempt to provide follow-up until a satisfactory clinical resolution of the laboratory result or adverse event is achieved.

Subjects who meet any of the following conditions will be discontinued from the study regardless of any causality assessment:
If the microneurographic recording is lost before oral administration or within 1 hour after oral dosing of study medication

### Individual Subject Stopping Criteria for Lidocaine Treatment

If there is occurrence of life-threatening AEs or if any of the following toxicities occur and potentially related to the lidocaine infusion, the infusion will be stopped immediately and the subject should be discontinued from the study:

Visual disturbances, dissociation, hallucinations, tremor, muscle twitching, bradycardia, hypotension, arrhythmias, agitation, convulsions, coma, respiratory arrest and cardiovascular collapse.
1. If the following toxicities occur and they are considered clinically significant and potentially related to the lidocaine infusion, the infusion rate will be reduced or stopped at the discretion of the anaesthetist and investigators:
   Hypersensitivity, circumoral paraesthesia, numbness of the tongue or tingling, dizziness, auditory disturbancies, confusion, drowsiness, increase in blood pressure, nausea and headache.
2. If any subject experiences clinically significant moderate AEs that are not mentioned above or unexpected based on the SPC of lidocaine but potentially related to the lidocaine infusion, the infusion rate will be decreased or stopped at the discretion of the anaesthetist and investigators. The following situations should be considered before reducing or stopping lidocaine infusion:
   a. The moderate AEs are considered unlikely related to lidocaine and e.g., considered to be related to concomitant disease or concomitant medication.
   b. The moderate AEs are due to procedures and do not represent a significant safety risk to the subject.
   c. The moderate AEs are commonly observed and therefore expected in early phase clinical trials where a relationship to the trial treatment or medication is unlikely but cannot be fully excluded and which do not present a significant clinical risk to the subjects such as pre-syncope, vasovagal reaction, anxiety, headache and nausea.
      In situations of i, ii or iii, the infusion may continue at the discretion of the investigator. For unblinding information please refer to Section 4.0.

### Lidocaine Group Stopping Criteria

If clinically significant lidocaine related severe adverse events are observed in two or more subjects, dosing in the lidocaine group will be suspended.

If there is occurrence of a clinically significant lidocaine related life threatening AE or a lidocaine related SAE, dosing in the lidocaine group will be suspended.

### Compound A Group Stopping Criteria

For Compound A, there are no adverse drug reactions identified in the current clinical program and no clinically important safety findings were observed in the Phase 1 safety, tolerability, and pharmacokinetic studies conducted in healthy adults and healthy elderly subjects and in the ongoing Phase 2 efficacy and safety study. In this study subjects with diabetic neuropathic pain were administered 100 mg BID up to 6 weeks and dosing has been completed. Nevertheless, if there is any new safety signal observed during this study, dosing of Compound A can be discontinued based on the joint decision of the investigators and study sponsor's medical monitor.

### 3.5 Discontinuation of Entire Study

Study sponsor may terminate this study prematurely, either in its entirety or at any study site, for reasonable cause provided that written notice is submitted in advance of the intended termination. The investigator may also terminate the study at his/her site for reasonable cause, after providing written notice to study sponsor in advance of the intended termination. Advance notice is not required by either party if the study is stopped due to safety concerns. If study sponsor terminates the study for safety reasons, study sponsor will immediately notify the investigator by telephone and subsequently provide written instructions for study termination.

### 4.0 Treatments

### 4.1 Treatments Administered

Study drug will be administered in the morning on Study Day 1 as follows:
Regimen A 100 mg Compound A (two 50 mg capsules, oral), IV placebo (glucose)
Regimen B 3 mg/kg Lidocaine IV infused over 30 minutes, 2 placebo capsules for Compound A (oral)
Regimen C Placebo (2 placebo capsules for Compound A, (oral), IV placebo (glucose)

Each oral dose of study drug will be taken with enough water to aid in swallowing of study medication. Study drug administration will be recorded to the nearest minute. The subjects will be instructed to remain in a semi-recumbent position until after the microneurography recording is completed.

### 4.2 Identity of Investigational Products

Information about the Compound A formulations to be used in this study is presented in Table 4.

TABLE 4
Identity of Investigational Products

| | Compound | | | |
|---|---|---|---|---|
| Investigational | A | | | |
| Products | 50 mg | Placebo | Lidocaine | Glucose |
| Dosage Form | Capsule | Capsule | Ampules | Bags of 50, |
| | (Size 00) | (Size 00) | 2 ml, 5 ml, | 100, 150, |
| | | | 10 mL or | 250, 500 or |
| | | | 20 mL | 1000 mL |
| Mode of | Oral | Oral | IV | IV |
| Administration | | | | |
| Strength (mg) | 50 | 0 | 2% w/v | Glucose |
| | | | | Monohydrate |
| | | | | for Parenteral |
| | | | | Use BP |
| | | | | 5.5% w/v |
| Manufacturer | Abbott | Abbott | Goldshield, | Fresenius Kabi |
| | | | UK | Limited, UK |

### 4.3 Packaging and Labeling

Study drug (Compound A and matching placebo) in capsule form will be provided in high density polyethylene bottles containing 30 capsules. Each bottle will be labeled as required per country requirement. Labels must remain affixed to the bottle.

### 4.4 Storage and Disposition of Study Drugs

### Study Drug for Oral Use

Compound A and placebo must be stored at 15° C. to 25° C. (59° F. to 77° F.) in a controlled storage area that should have a temperature recording device. A storage temperature log is to be maintained to document proper storage conditions. The temperature must be recorded every business day. Malfunctions or any temperature excursions must be reported to study sponsor immediately. In case of a temperature excursion, study drug should be quarantined and not dispensed until study sponsor deems the medication as acceptable. The investigational products are for investigational use only and are to be used only within the context of this study. The study drug supplied for this study must be maintained under adequate security and stored under the conditions specified on the label until dispensed for subject use or destroyed locally.

### Comparator Drug for IV Use

The lidocaine and glucose must be stored according to conditions specified on the original package information in a controlled storage area that should have a temperature recording device. A storage temperature log is to be maintained to document proper storage conditions. The temperature must be recorded every business day. Malfunctions or any temperature excursions must be reported to Study sponsor immediately. In case of a temperature excursion, study drug must be damaged and replaced. The products are to be used only within the context of this study. The product for this study must be maintained under adequate security and stored under the conditions specified on the label until dispensed for subject use or destroyed locally.

### 4.5 Method of Assigning Subjects to Treatment Groups

As they are enrolled in the study, subjects will be assigned unique consecutive numbers beginning with 101. The subjects will be randomly assigned to a regimen as shown in Section 1.1. The randomization schedule will be computer-generated before the start of the study by the Statistics Department of study sponsor.

### 4.6 Selection and Timing of Dose for Each Subject

Selection of the dose for this study is discussed in Section 5.2. The same dose will be administered to all subjects. Dosing will be accomplished on Study Day 1.

### 4.7 Blinding

The study will be conducted in a double-blind manner such that the investigator and subjects are blinded to the treatment assignments. All study site personnel, except for the pharmacist, will remain blinded to the treatment. The pharmacist will receive a copy of the randomization codes in a sealed envelope prior to the start of the study and will maintain the randomization codes and individual drug accountability logs in a restricted and secured location separate from the overall site drug accountability logs. The unblinded pharmacist at the study site will prepare the individual subject doses.

The Compound A capsules and placebo capsules will be identical in appearance and will be maintained under the same storage conditions. The lidocaine and glucose packs will be identical in appearance. A document that contains the study drug assignment will be provided to the investigator in a separate sealed envelope for each subject. Study sponsor must be notified before breaking the blind, unless identification of the study drug is required for emergency therapeutic measures. The study blind envelope may be opened if, in the opinion of the investigator, it is in the subject's best interest or necessitated for safety reasons.

If study sponsor is not informed of the blind break prior to the blind break occurring, Study sponsor must then be notified within 24 hours of the blind being broken. The date, time and the initials of the person who opened the blind-breaker envelope will be recorded along with the reason for the blind break. Once the study is complete, all blind-breaker envelopes (sealed and opened) must be inventoried and returned to study sponsor.

### 5.0 Treatment Compliance

The investigator or his/her designated and qualified representatives will administer study drug only to subjects enrolled in the study in accordance with the protocol. The study drug must not be used for reasons other than that described in the protocol. Subjects will be confined and supervised at the time of study drug administration. Study-site personnel will perform hand and mouth checks to ensure ingestion of each dose.

### 5.1 Drug Accountability

The investigator or his/her representative will verify that study drug supplies are received intact and in the correct amounts. This will be documented by signing and dating the Proof of Receipt or similar document. The investigator or his/her designated representatives will administer study drug only to subjects enrolled in the study. A current (running) and accurate inventory of study drug will be kept by the investigator and will include shipping invoices and the date on which study drug is administered to the subject. An overall accountability of the study drug will be performed and verified by study sponsor monitor throughout the study and at the study site closeout visit. Upon completion or termination of the study, all original containers (empty or containing unused study drug) will destroyed locally, according to instructions from study sponsor and according to local regulations. Labels must remain attached to the containers.

### 5.2 Discussion and Justification of Study Design

### Discussion of Study Design and Choice of Control Groups

This is a randomized, double-blind, placebo-controlled, parallel-group, single center study. Double-blind and placebo-controlled designs are generally acknowledged as standard for unbiased estimates of treatment group differences. Parallel-group is used in this study because C-nociceptors that are able to be detected and identified in each microneurographic recording vary significantly within-subject. Lidocaine, a sodium channel blocker, is believed to block hyperactivity of sodium channels present in damaged nociceptors. Systemic administration of lidocaine has shown analgesic effects in several neuropathic pain conditions. Lidocaine therefore is included in the study to assess its effect on spontaneous activity of C-nociceptors and is expected to provide information of correlation between spontaneous activity of C-nociceptors and clinical efficacy.

### Appropriateness of Measurements

Microneurography will be used to assess the pharmacodynamic effect of study drugs on spontaneous activity of C-nociceptors. Microneurography is a technique in which the activity of single nerve fibers is recorded from peripheral nerves and it is the only technique for directly detecting and quantifying the function of pain fibers in peripheral nerves in humans. In addition, standard pharmacokinetic, clinical and laboratory procedures will be utilized in this study.

### Suitability of Subject Population

Diabetic neuropathic pain is mainly described as continuous or intermittent (spontaneous) burning, aching, throbbing pain. Spontaneous pain may attribute to ectopic activity in C-nociceptors. In pathological states, C-nociceptors can become hyperexcitable and generate spontaneous ectopic discharges. There is evidence that C-nociceptors are more frequently spontaneously active in neuropathic pain patients than in healthy subjects (Ochoa J L, Campero M, Serra J, Bostock H. Hyperexcitable polymodal and insensitive nociceptors in painful human neuropathy. Muscle and Nerve. 2005; 32:459-472). Therefore, assessing spontaneous activity in patients with diabetic neuropathic pain may provide a direct link between activity in peripheral nerve fibers and clinical pain perception.

### Selection of Doses in the Study

Single doses of Compound A up to 170 mg were well-tolerated in healthy subjects. The Compound A dose of 100 mg twice daily (BID) is the clinical dose used in the ongoing Phase 2a study in subjects with diabetic neuropathic pain. The dose of 100 mg is selected in the current study to explore the effect of Compound A in a microneurography model in order to bridge the efficacy outcome of Compound A in the Phase 2a study.

Lidocaine IV infusion with 2 mg/kg or 5 mg/kg IV has shown reduction of pain in patients with painful diabetic neuropathy while the dose of 5 mg/kg demonstrated consistent superiority to placebo in pain relief Based on the available data and benefit/risk profile of IV infusion of lidocaine from clinical studies, 3 mg/kg was selected for infusion in this study.

### 6.0 Adverse Events

The investigator will monitor each subject for clinical and laboratory evidence of adverse events on a routine basis throughout the study. The investigator will assess and record any adverse event in detail including the date of onset, event diagnosis (if known) or sign/symptom, severity, time course, outcome, relationship of the adverse event to study drug, and any action(s) taken. For serious adverse events not considered “probably related” to study drug, the investigator will provide an Other cause of the event. For adverse events to be considered intermittent, the events must be of similar nature and severity. Adverse events, whether in response to a query, observed by site personnel, or reported spontaneously by the subject will be recorded.

All adverse events will be followed to a satisfactory conclusion.

### 6.1 Definitions

### Adverse Event

An adverse event is defined as any untoward medical occurrence in a patient or clinical investigation subject administered a pharmaceutical product and which does not necessarily have a causal relationship with this treatment. An adverse event can therefore be any unfavorable and unintended sign (including an abnormal laboratory finding), symptom, or disease temporally associated with the use of a medicinal (investigational) product, whether or not the event is considered causally related to the use of the product.

Such an event can result from use of the drug as stipulated in the protocol or labeling, as well as from accidental or intentional overdose, drug abuse, or drug withdrawal. Any worsening of a pre-existing condition or illness is considered an adverse event. Worsening in severity of a reported adverse event should be reported as a new adverse event. Laboratory abnormalities and changes in vital signs are considered to be adverse events only if they result in discontinuation from the study, necessitate therapeutic medical intervention, and/or if the investigator considers them to be adverse events.

An elective surgery/procedure scheduled to occur during a study will not be considered an adverse event if the surgery/procedure is being performed for a pre-existing condition and the surgery/procedure has been pre-planned prior to study entry. However, if the pre-existing condition deteriorates unexpectedly during the study (e.g., surgery performed earlier than planned), then the deterioration of the condition for which the elective surgery/procedure is being done will be considered an adverse event.

### 6.2 Serious Adverse Events

If an adverse event meets any of the following criteria, it is to be reported to study sponsor as a serious adverse event within 24 hours of the site being made aware of the serious adverse event:

| | |
|---|---|
| Death of Subject | An event that results in the death of a subject. |
| Life-Threatening | An event that, in the opinion of the investigator, would have |
| | resulted in immediate fatality if medical intervention had not |
| | been taken. This does not include an event that would have |
| | been fatal if it had occurred in a more severe form. |
| Hospitalization or | An event that results in an admission to the hospital for any |
| Prolongation of | length of time or prolongs the subject's hospital stay. This does |
| Hospitalization | not include an emergency room visit or admission to an |
| | outpatient facility. |
| Congenital Anomaly | An anomaly detected at or after birth, or any anomaly that |
| | results in fetal loss. |
| Persistent or Significant | An event that results in a condition that substantially interferes |
| Disability/Incapacity | with the activities of daily living of a study subject. Disability |
| | is not intended to include experiences of relatively minor |
| | medical significance such as headache, nausea, vomiting, |
| | diarrhea, influenza, and accidental trauma (e.g., sprained ankle). |
| Important Medical Event | An important medical event that may not be immediately life- |
| Requiring Medical or | threatening or result in death or hospitalization, but based on |
| Surgical Intervention to | medical judgment may jeopardize the subject and may require |
| Prevent Serious | medical or surgical intervention to prevent any of the outcomes |
| Outcome | listed above (i.e., death of subject, life-threatening, |
| | hospitalization, prolongation of hospitalization, congenital |
| | anomaly, or persistent or significant disability/incapacity). |
| | Examples of such events include allergic bronchospasm |
| | requiring intensive treatment in an emergency room or at home, |
| | blood dyscrasias or convulsions that do not result in inpatient |
| | hospitalization, or the development of drug dependency or drug |
| | abuse. |
| Spontaneous Abortion | Miscarriage experienced by study subject. |
| Elective Abortion | Elective abortion performed on study subject. |

For serious adverse events with the outcome of death, the date and cause of death will be recorded on the appropriate case report form.

### 6.3 Adverse Event Severity

The investigator will use the following definitions to rate the severity of each adverse event:

| | |
|---|---|
| Mild | The adverse event is transient and easily tolerated by the |
| | subject. |
| Moderate | The adverse event causes the subject discomfort and |
| | interrupts the subject's usual activities. |
| Severe | The adverse event causes considerable interference with the |
| | subject's usual activities and may be incapacitating or |
| | life-threatening. |

### 6.4 Relationship to Study Drug

The investigator will use the following definitions to assess the relationship of the adverse event to the use of study drug:

| | |
|---|---|
| Probably | An adverse event has a strong temporal relationship to study |
| Related | drug or recurs on re-challenge and an Other cause of event |
| | is unlikely or significantly less likely. |
| Possibly | An adverse event has a strong temporal relationship to the |
| Related | study drug and an Other cause of event is equally or less |
| | likely compared to the potential relationship to study drug. |
| Probably | An adverse event has little or no temporal relationship to the |
| Not | study drug and/or a more likely Other cause of event exists. |
| Related | |
| Not | An adverse event is due to an underlying or concurrent illness |
| Related | or effect of another drug and is not related to the study drug |
| | (e.g., has no temporal relationship to study drug or has a |
| | much more likely Other cause of event). |

For causality assessments, events meeting the categories of probably or possibly will be considered “associated.” Events that are probably not or not related will be considered “not associated.” In addition, when the investigator has not reported a causality or deemed it not assessable, study sponsor will consider the event associated.

If an investigator's opinion of possibly, probably not, or not related to study drug is given, an Other cause of event must be provided by the investigator for the serious adverse event.

### 6.5 Adverse Event Collection Period

All adverse events reported from the time of study drug administration until 30 days following discontinuation of study drug administration have elapsed will be collected, whether solicited or spontaneously reported by the subject. In addition, serious adverse events will be collected from the time the subject signed the study-specific informed consent.

During the outpatient/washout portion(s) of the study, subjects will be provided with a telephone number for the study site and instructions to contact the study site if they experience an adverse event requiring medical care. Adverse event and medical history information will be updated upon subject reconfinement to confirm eligibility for continued participation in the study.

Adverse event information will be collected as shown in below.

### Adverse Event Collection

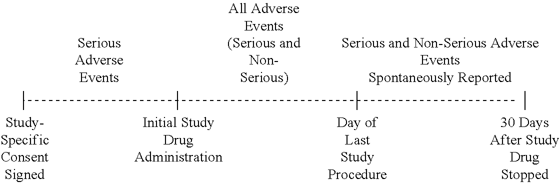

### 6.6 Adverse Event Reporting

In the event of a serious adverse event, whether related to study drug or not, the investigator will notify the Pain Care Management Team within 24 hours of the site being made aware of the serious adverse event by faxing the appropriate serious adverse event forms to the Pain Care Safety Team.

The sponsor will be responsible for Suspected Unexpected Serious Adverse Reactions (SUSAR) reporting for the Investigational Medicinal Product (IMP) in accordance with Directive 2001/20/EC. The reference document used for SUSAR reporting in the EU countries will be the most current version of the Investigator's Brochure. The reference document used for SUSAR reporting in the EU countries will be the most current version of the Summary of Product Characteristics (SmPC).

### 6.7 Pregnancy

Pregnancy in a study subject must be reported to study sponsor within 1 working day of the site becoming aware of the pregnancy to one of the study sponsor's representatives listed in Section 6.4. Subjects who become pregnant during the study must be discontinued.

Information regarding a pregnancy occurrence in a study subject and the outcome of the pregnancy will be collected.

Pregnancy in a study subject is not considered an adverse event. However, the medical outcome of an elective or spontaneous abortion, stillbirth or congenital anomaly, is considered a serious adverse event and must be reported to study sponsor within 24 hours of the site becoming aware of the event.

### 6.8 Risk Management

Compound A is a highly selective T-type (Cav3.2) calcium channel blocker. The safety, tolerability, and pharmacokinetics of Compound A in humans have been evaluated in 94 healthy adult subjects in the Phase 1 first-in-human (FIH) study with the single dose up to 170 mg and multiple doses up to 160 mg for 7 to 14 days. The incidence of adverse events was similar across doses and occurrence of adverse events does not appear to be dose-dependent. All adverse events were mild except one event. Compound A was well tolerated. There are no clinically relevant findings observed in vital signs, ECG and laboratory tests. Given that Compound A is an investigational drug and this study will be conducted in diabetic patient population that generally has high risk factors, inclusion and exclusion have been carefully established to avoid exposing patients to undue risk. In addition, adequate safety monitoring procedures and stopping criteria are placed in the protocol.

Microneurography is a safe technique if performed by adequately trained personnel. There have been no reports of overt or persistent nerve damage, and prospective studies monitoring side effects of the technique have proved it to be safe. There have been no reports of local infection or bleeding. Normally, subjects will experience no side effects after the procedure. In a minority of occasions, subjects may experience a sensation of tingling for a few days after the procedure if tapping over the nerve. In this study, microneurography will be performed by a highly trained investigator. If abnormal sensation develops after the procedure, the subject will be followed until the event is resolved and medical evaluation and treatment will be provide as appropriate.

An 8-hour fasting is required for study administration. To avoid hypoglycemia condition, the subject's blood glycemic level will be closely monitored prior to, during the procedure and post dosing. A light high carbohydrate snack may be provided to the subject as appropriate at the investigator's discretion based on glycemic levels.

Intravenous lidocaine infusions have long been used to treat severe pain, including neuropathic pain, both under experimental and clinical conditions. Doses administered in the referenced clinical trials were 5 mg/kg body weight over 30 minutes (See, Attal N, Rouaud J, Brasseur L. Systemic lidocaine in pain due to peripheral nerve injury and predictors of response. Neurology. 2004; 62:218 and Attal N, Gaude V, Brasseur L. Intravenous lidocaine in central pain: a double-blind, placebo-controlled, psychophysical study. Neurology. 2000; 54:564) or a bolus of 1 mg/kg followed by an infusion of 4 mg/kg over 40 minutes (Wu C L. Analgesic effects of intravenous lidocaine and morphine on postamputation pain: a randomized double-blind, active placebo-controlled, crossover trial. Anaesthesiology. 2022; 96:841-48). In therapeutic use various treatment regimens are available. A lidocaine test may be conducted with 1 to 3 mg/kg lidocaine as an 8 mg/mL infusion over 20 to 30 minutes. If this test is effective or partially effective, then continuous maintenance infusions may range between 0.5 to 2 mg/kg per hour and titrated to minimize the lidocaine dose whilst controlling the pain (See, Ferrini R, Paice J A. How to initiate and monitor infusional lidocaine for severe and/or neuropathic pain. The Journal of Supportive Oncology. 2004; 2(1):90-4).

The side effect profile of parenteral lidocaine is well established and predictable. Lidocaine has a short half-life and therefore the symptoms of lidocaine toxicity are transient and reversible by lowering the infusion rate or discontinuing the infusion. Lidocaine specific stopping rules outlined in Section 3.3. specify how potential lidocaine toxicities will be dealt with in terms of dosing.

Continuous cardiac monitoring via telemetry will be performed during the infusion of lidocaine/placebo. The patient will be supervised by an anaesthetist during the infusion until at least one hour after the end of infusion.

The guidelines of the Association of Anaesthetists of Great Britain and Ireland (AAGBI) for the ‘Management of Severe Local Anaesthetic Toxicity’ and ‘Accompanying Notes’ will be followed for the treatment of severe lidocaine toxicity.

This study will be conducted in a specialized early phase CPU within an acute hospital setting with Critical Care facilities, thus ensuring direct access to equipment and staff for resuscitating and stabilizing subjects in acute medical conditions and emergencies. The study is conducted by experienced investigators and well trained medical, nursing and technical staff with ample experience in the conduct of early phase clinical trials.

The study is designed to closely monitor, treat and communicate potential expected adverse reactions (based on the known mode of action of the non-IMP and the previous studies) as well as potential unexpected AEs. Patients may experience therapeutic benefit from the infusion of lidocaine. Pain relief following lidocaine infusion may be effective for up to 21 days in patients with diabetic neuropathy. Based upon the mechanism of action and the data from animal pain models, Compound A may have an effect on decreasing spontaneous activity of the C-fiber which could potentially reduce pain.

### 6.9 Protocol Deviations

The investigator should not implement any deviation from the protocol without prior review and agreement by the Sponsor and in accordance with the IEC/IRB and local regulations, except when necessary to eliminate an immediate hazard to study subjects. When a deviation from the protocol is deemed necessary for an individual subject, the investigator must contact the study sponsor.

Such contact must be made as soon as possible to permit a review by study sponsor to determine the impact of the deviation on the subject and/or the study. Any significant protocol deviations affecting subject eligibility and/or safety must be reviewed and/or approved by the IEC/IRB and regulatory authorities, as applicable, prior to implementation.

### 7.0 Statistical Methods and Determination of Sample Size Statistical and Analysis Plans

### Demographics

Descriptive statistics will be provided with a breakdown by regimen for demographic variables and baseline values of variables of interest.

### Pharmacokinetics

Plasma concentrations of Compound A and lidocaine and pharmacokinetic parameter values will be tabulated for each subject and each regimen, and summary statistics will be computed for each sampling time and each parameter.

### Safety

Adverse events will be coded using the Medical Dictionary for Regulatory Activities (MedDRA). The number and percentage of subjects reporting treatment-emergent adverse events will be tabulated by MedDRA preferred term and system organ class (SOC) with a breakdown by regimen. Laboratory test values, measurements of vital signs, and ECG measurements that are potentially clinically significant, according to predefined criteria, will be identified.

Additional analyses will be performed if useful and appropriate.

Pharmacodynamics

For each of the microneurography variables, descriptive statistics will be provided by regimen and time interval (baseline and each 10-minute interval after oral dosing and initiation of infusion). For a post dose time interval, the descriptive statistics will be provided for the time interval itself and for the change from baseline. The descriptive statistics will include an estimate and test on the mean difference from baseline that is obtained in the framework of a model in which the fibers of a subject from which measurements are taken are viewed as a random sample from the subject.

For each variable, the data for the 10 minute intervals will be jointly analyzed using a linear mixed effects model. The model will include classification of observations by regimen, time interval and the interaction of regimen and time interval. The effects defined by these classifications will all be fixed. The baseline value will also be included as a covariate. The subjects will be viewed as a random sample. If there are data for more than one fiber of a subject, the fibers will be considered a random sample from the subject. An appropriate structure for the variance/covariance matrix for the observations from a subject will be selected. Within the framework of the model the effects (differences in mean from placebo) of Compound A and lidocaine will be estimated for each time interval and for the average over all the time intervals. In association with these estimates of the effects of Compound A results of hypothesis tests will be reported, and the same may be done for lidocaine.

The length of time that subjects provide microneurography recordings is expected to vary. If there are little data for later time intervals, an analysis may be performed on a data set that includes data only up to a certain time point. However, in that case other analyses may be performed that include the data of the later time intervals. In associations with these estimates of the effects of Compound A, results of hypothesis tests will be reported, and the same may be done for lidocaine.

If it appears that a variable has a probability distribution with substantial nonsymmetry, a transformation may be employed in order to have an approximately symmetric distribution.

Descriptive statistics with a breakdown by regimen will be provided for numerical rating score (NRS) for pain at baseline and for each time post dose.

Relationship of pain intensity and spontaneous activity in C-nociceptors will be explored.

### Determination of Sample Size

No formal sample size calculations are performed for this study. It is expected that 20 to 25 fibers (1 to 3 fibers from each subject) for each treatment group will be adequate to characterize and assess spontaneous activity in C-nociceptors and assess treatment effects.

### 8.0 Ethics

### Independent Ethics Committee (IEC) or Institutional Review Board (IRB)

Good Clinical Practice (GCP) requires that the clinical protocol, any protocol amendments, the Investigator's Brochure, the informed consent and all other forms of subject information related to the study (e.g., advertisements used to recruit subjects) and any other necessary documents be reviewed by an IEC/IRB. The IEC/IRB will review the ethical, scientific and medical appropriateness of the study before it is conducted. IEC/IRB approval of the protocol, informed consent and subject information and/or advertising, as relevant, will be obtained prior to the authorization of drug shipment to a study site.

Any amendments to the protocol will require IEC/IRB approval prior to implementation of any changes made to the study design. The investigator will be required to submit, maintain and archive study essential documents according to ICH GCP.

Serious adverse events that meet the reporting criteria, as dictated by local regulations, will be reported to both responsible Ethics Committees and Regulatory Agencies as required by local regulations. During the conduct of the study, the investigator should promptly provide written reports (e.g., ICH Expedited Reports or any additional reports required by local regulations) to the IEC/IRB of any changes that affect the conduct of the study and/or increase the risk to subjects. Written documentation of the submission to the IEC/IRB should also be provided to study sponsor.

### Ethical Conduct of the Study

The study will be conducted in accordance with the protocol, International Conference on Harmonization (ICH) GCP guidelines, applicable regulations and guidelines governing clinical study conduct and ethical principles that have their origin in the Declaration of Helsinki.

### Subject Information and Consent

Prior to the initiation of any screening or study-specific procedures, the investigator or his/her representative will explain the nature of the study to the subject and answer all questions regarding this study. Each informed consent will be reviewed, signed and dated by the subject, the person who administered the informed consent, and any other signatories according to local requirements. A copy of each informed consent will be given to the subject and each original will be placed in the subject's medical record. An entry must also be made in the subject's dated source documents to confirm that informed consent was obtained prior to any study-related procedures and that the subject received a signed copy.

### Source Documents and Case Report Form Completion

### Source Documents

Source documents are defined as original documents, data, and records. These may include hospital records, clinical and office charts, laboratory data/information, subject diaries or evaluation checklists, pharmacy dispensing and other records, recorded data from automated instruments, microfiches, photographic negatives, microfilm or magnetic media, and/or x-rays. Source document data may be transcribed onto case report forms (CRFs) as required. Data collected during this study must be recorded on the appropriate source document.

The investigator/institution will permit study-related monitoring, audits, IEC/IRB review, and regulatory inspection(s), providing direct access to source data documents.

### Case Report Forms

Case report forms will be supplied by study sponsor. These forms will be used to transmit information collected during this study to study sponsor and regulatory authorities, as applicable. Case report forms must be completed for each subject enrolled in this study. All case report forms must be legible and completed in indelible ballpoint ink. Any necessary corrections are to be made by drawing a single line through the incorrect entry and writing in the revision, the date of the correction, the reason for the correction, and the initials of the person making the correction (i.e., principal investigator or his/her representative). Data are not to be obliterated by blacking out, using correction fluid or by erasing the original entry. All information written on the case report forms must also be reflected in the subject source documents.

The chief investigator will review the case report forms for completeness and accuracy and sign and date the set of case report forms where indicated. Study sponsor personnel (or their representatives) will review the case report forms periodically for completeness, legibility, and acceptability. Study sponsor (or their representatives) will be allowed access to all source documents in order to verify case report form entries.

Each case report form will be printed on two-part no-carbon-required paper. The white original will be sent to study sponsor and the pink copy will remain at the study site. The original signed case report form and a copy of the laboratory reports will be collected. A copy of the case report form will remain at the study site. Once the original case report form has been separated/removed from the study site, all changes must be made via the appropriate change form specified by study sponsor. The principal investigator will review the change form for completeness and accuracy and sign and date the change form where indicated.

### Data Quality Assurance

Prior to enrolling any subject in the study, an initiation meeting will be held with study sponsor personnel, the investigator(s), and the study coordinators/project manager(s). This meeting will include a detailed discussion and review of the protocol and essential documents, performance of study procedures, case report form completion and specimen collection methods.

The study sponsor monitor will monitor the study site throughout the study. A 100% source document review will be made against entries on the case report forms and a quality assurance check will be performed to ensure that the investigator is complying with the protocol and regulations. In addition, after the case report forms are retrieved, a review of the data will be conducted by a physician or representative at study sponsor.

After completion of the entry process, computer logic checks will be run to identify such items as inconsistent study dates. Any necessary corrections will be made to the database via the electronic CRF.

Routine hematology, serum chemistry and serology, and urinalysis tests will be conducted using a certified clinical laboratory. Laboratory reference ranges will be obtained prior to the initiation of the study. A review of all laboratory results will be conducted by the study sponsor monitor, the investigator and other appropriate personnel from study sponsor.
The end of study is defined as the date of the last subject's last visit.

All references (patent and non-patent) cited above are incorporated by reference into this patent application. The discussion of those references is intended merely to summarize assertions made by their authors. No admission is made that any reference (or a portion of a reference) is relevant prior art (or prior art at all). Applicants reserve the right to challenge the accuracy and pertinence of the cited references.

We claim:

1. A method for treating diabetic neuropathy in a human subject in need of such treatment, wherein the method comprises administering to the human subject an amount of therapeutic agent A or a salt thereof:

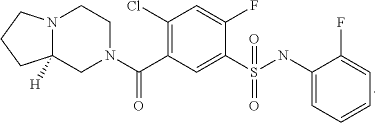 [SMILES: O=C(c1cc(S(=O)(=O)Nc2ccccc2F)c(F)cc1Cl)N1CCN2CCC[C@@H]2C1]

2. The method of claim 1, wherein therapeutic agent A is a salt of compound A.

3. The method of claim 1, wherein the diabetic neuropathy is diabetic peripheral neuropathy.

4. The method of claim 1, wherein the amount of compound A is from about 1 to about 500 mg per day.

5. The method of claim 1, wherein the amount of compound A is from about 10 to about 100 mg per day.

6. The method of claim 1, wherein therapeutic agent A is administered orally.

7. The method of claim 6, wherein therapeutic agent A is administered in a solid dosage form.

8. The method of claim 1, wherein the method further comprises monitoring the treatment in humans using microneurography.
